(19) United States
(12) Patent Application Publication
(10) Pub. No.: US 2024/0418593 A1
(43) Pub. Date: Dec. 19, 2024

(54) EMBEDDED PRESSURE SENSING SYSTEM FOR INTERNAL BLAST PRESSURE MEASUREMENTS

(71) Applicant: The United States of America as Represented by the Secretary of the Navy, Silver Spring, MD (US)

(72) Inventors: Luke Beardslee, Atlanta, GA (US); Michael K. Qin, Fairfax, VA (US); Brandon M. Casper, Waterford, CT (US); Joshua Todd, Tolland, CT (US)

(73) Assignee: The United States of America as Represented by the Secretary of the Navy, Silver Spring, MD (US)

(21) Appl. No.: 17/743,245

(22) Filed: May 12, 2022

Related U.S. Application Data
(60) Provisional application No. 63/187,499, filed on May 12, 2021.

(51) Int. Cl. G01L 23/10; A61B 5/00; A61B 5/07

(52) U.S. Cl. CPC G01L 23/10; A61B 5/073; A61B 5/076; A61B 5/6861; A61B 2560/0209

(57) ABSTRACT

The present invention relates to a device and method for monitoring internal pressures and pressure changes of a subject resulting from a blast. In particular, the present invention relates to an ingestible capsule, which when swallowed and passed through the subject, can be used to capture, record and transfer information including internal blast pressures experienced by the subject.

### CROSS REFERENCE TO RELATED APPLICATIONS

This application claims priority to U.S. Provisional Application No. 63/187,499 filed on May 12, 2021, which is hereby incorporated by reference.

### FEDERAL SPONSORED RESEARCH

This invention was made with government support under N62645-18-D-5049 awarded by Department of the Navy. The government has certain rights in the invention.

### FIELD OF THE INVENTION

This present invention relates to a device and a method for monitoring internal pressure experienced by an individual caused by a blast from an improvised explosive device. In particular, the present invention relates to a blast capsule, which when swallowed and passed through the digestive tract of a subject, can measure, record and transmit information about internal pressure experienced by the subject from a blast.

### BACKGROUND

The Explosive Ordnance Disposal (EOD) and Special Operations Forces (SOF) communities are required to work with and around explosives as part of their daily operation. While established explosive ordnance disposal (EOD) guidance documentation sets safe standoff distances from explosives, it fails to provide specific information on probability of injury, and type and severity of injury should an unanticipated explosion occur. Such information is critical for mission planners to evaluate operational risk, and to improve operational effectiveness. In an operational setting, injury prevention requires a balance between keeping service members safe and mission accomplishment.

Wearable blast sensing technologies have been of interest across several defense agencies. One example is the Blast Gauge System, which is developed by BlackBox Biometrics Incorporated, and used to detect possible physiological and psychological trauma caused by exposures to various amounts of blast pressure. The system consists of a helmet sensor and two torso sensors, each of which communicate wirelessly to give a full-body scope of the pressures to which the wearer is exposed. Following field implementation, it is found to positively affect operational readiness, which allowed detection of conditions that would otherwise be unrecognized and undiagnosed

Naval warfare in the littorals has much in common with war conducted on the open ocean and on land, and securing littoral battlespace is a major challenge that the U.S. Navy faces in the 21^{st} century. While every effort is made to prevent the unintended detonation of underwater explosion devices, land based personnel and U.S. Navy divers can be exposed to high-energy underwater explosions. Methods are needed to characterize the internal pressures experienced by divers caused by shallow water blasts. These data can be combined with numerical models to develop new guidance about safe stand-off distances for searching, examining, and diffusing charges in littoral waters. Information on injury mechanisms and blast dosage is also important for first responders to provide optimal, targeted care to patient with blast injuries. As many as half of all injuries in recent overseas operations resulted from explosions. Air filled structures, such as intraabdominal organs, are particularly susceptible to injury from blast. Identifying patients with internal injuries quickly and rapidly getting them to surgical care is essential to improving patient outcomes.

While the current wearable blast sensors are water resistant, they are not designed to be regularly exposed to water or be submerged in fluid for an extended period. Without a viable underwater blast sensor, previous studies have attempted to indirectly examine the effects of underwater blast on living tissue. Some studies tried to characterize risks of underwater blast by measuring the pressures created near a school of fish that was exposed to simulated underwater explosions. Others examined the effects of underwater explosion using cadavers implanted with pressure transducers. Both models are inadequate due to numerous assumptions and various intervening factors, which leave an incomplete picture of underwater blast on living tissue. For example, when conducting external pressure measurements near a school of fish, the researcher must take into account of the orientation, actual exposure, and individual variation of the fish. With cadaveric model, researchers must consider post mortem tissue changes, and the lack of muscle tone in cadavers. Gap in our knowledge about the internal pressures that organs may experience in the event of an underwater blast limits our ability to assess risk, predict injury, and develop safety and treatment guidance.

An embedded (i.e. ingestible or implantable) pressure sensing system would allow direct quantification of the pressures that an individual experienced from a blast, and correlates them with observed injuries. Various ingestible capsule systems have been developed and commercialized for medical applications. The PILICAM™ (Medtronic, Minneapolis, MN) takes diagnostic images of the small intestine and the colon. The VitalSense temperature capsule (Philips Inc. NV) travels and passes through the gastrointestinal tract of an animal, measures and transmits body temperature 4 times per minutes within 12-48 hour period. U.S. Pat. No. 7,647,090 to Firisch et al. in-vivo sensing capsule with interchangeable functional modules that measures temperature, in-vivo imaging, pH. The INTELLISITE® Capsule (Casper Associates, Sanford, NC) is a radiofrequency activated, non-disintegrating drug delivery device capable of non-invasive controlled delivery of drug formulations to the gastrointestinal tract for determining regional differences in drug absorption and bioavailability. A similar drug dispenser capsule is also described in U.S. Pat. No. 6,929,636 to von Alten. Other examples include the Bravo Capsule for pH measurement, the Vibrant Capsule (Vibrant Ltd, Hakochav, Yokneam, Isreal) to relieve constipation, and the Wireless Motility/pH Capsule (U.S. Pat. No. 8,945,010, Smartpill Corporation), which is used to gauge gastrointestinal motility. In addition to commercial devices, capsule technologies have been investigated for a variety of purposes including drug delivery, chemical sensing, tissue sampling/biopsy, bleeding detection, and a variety of additional applications, which are summarized in recent review papers exist on the topic.

Although ingestible capsule technologies exist, the integration of a blast sensor within an ingestable/implantable (embedded) capsule, and the electronics to operate the sensor have not been investigated. Designing an ingestible blast sensor presents distinct challenges because, unlike biological signals (such as gastro contraction) which tend to occur at lower frequency, blast or high-energy acoustic signals have higher frequency range, which require sampling rates as high as 1-2 megasample per second (MSPS). Design tradeoffs must be made between performance and power consumption to accommodate faster electric circuits that consume large amounts of power.

This invention meets this distinct challenge in designing an ingestible/implantable capsule containing a blast sensing system. The inventive blast capsule has the potential to enable better forward resuscitative care by identifying injuries associated with underwater or air blast and to improve blast surveillance by providing data on chronic blast exposure.

### DETAILED DESCRIPTION OF THE FIGURES

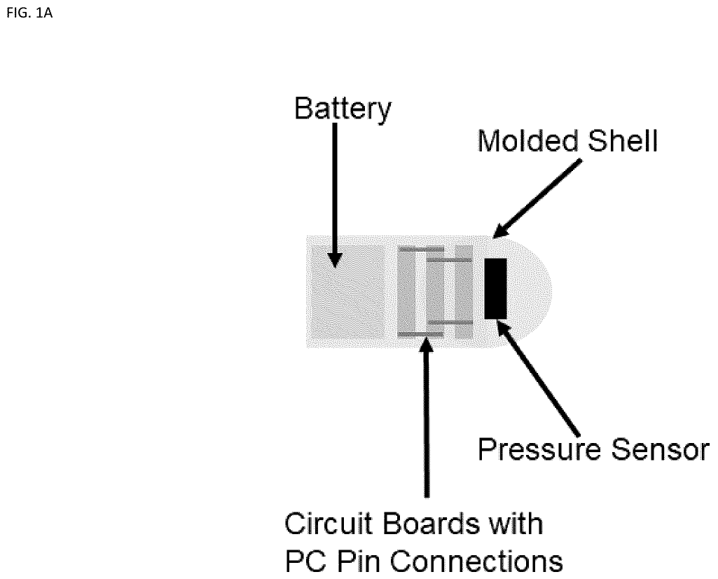

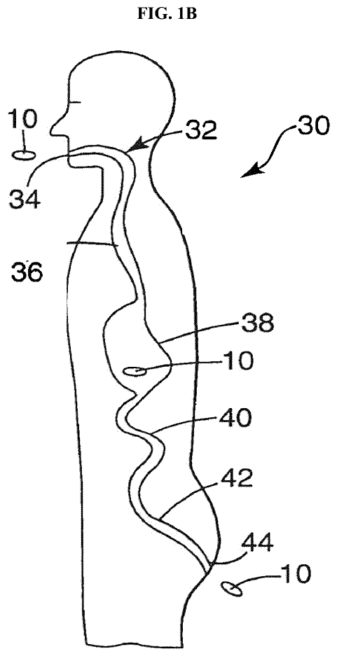

FIG. 1A is a schematic drawing of a prototype inventive blast sensor.

FIG. 1B is diagram view of a digestive tract of a human body schematically showing the travel path of an ingestible blast sensor, according to an embodiment of the present invention.

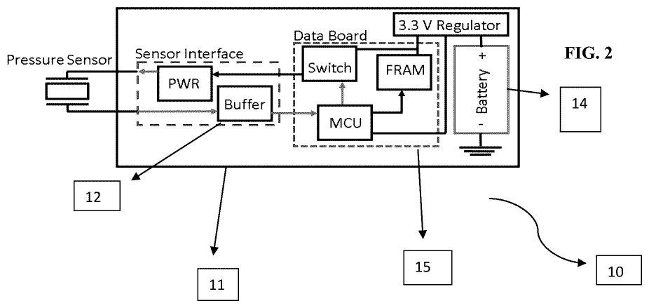

FIG. 2 is diagram of the electronics used to operate the capsule.

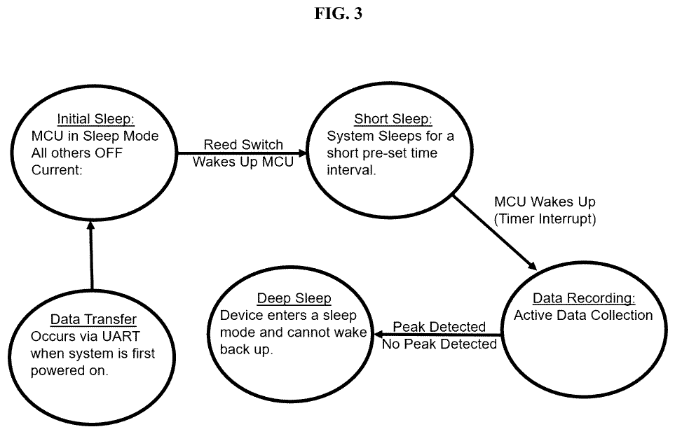

FIG. 3 is a block diagram outlining the operation of the blast capsule algorithm. This algorithm is programmed into the onboard microcontroller and takes the system through all of the necessary measurement steps.

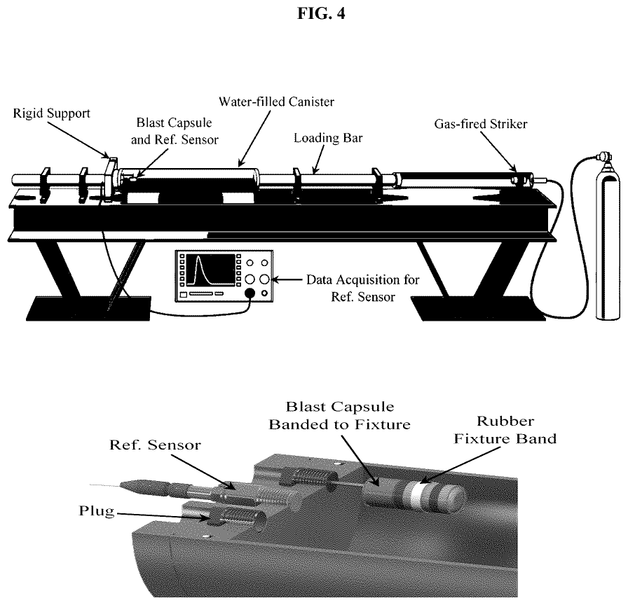

FIG. 4 shows the testing setup used for testing of the prototype blast capsule. Image of the testing setup used for testing of the prototype blast capsule. (Top) A gas fired projectile is launched using a solenoid operated gas reservoir charged between 75 and 100 PSI. The projectile strikes the Loading Bar launching a pressure wave in it. The Loading Bar is in contact with the water-filled canister that couples the pressure wave into the fluid. The canister measured 24″ in length, had an inner diameter of 3.75″ and an outer diameter of 4″. The canister's forward cap had a thickness of 1.25″ and the rear cap had a thickness of 1.875.″ The blast capsule and the reference sensor are at the far side of the canister. The reference sensor signal displayed using an oscilloscope (Tektronix DPO3034 Digital Phosphor Oscilloscope, Tektronix Inc., Beaverton, OR) set to peak capture and sampling at 250 MHz. (Bottom) A close-up of the reference sensor and blast capsule in its fixture at the rear cap of the vessel. The view is a horizontal section at a radial midplane.

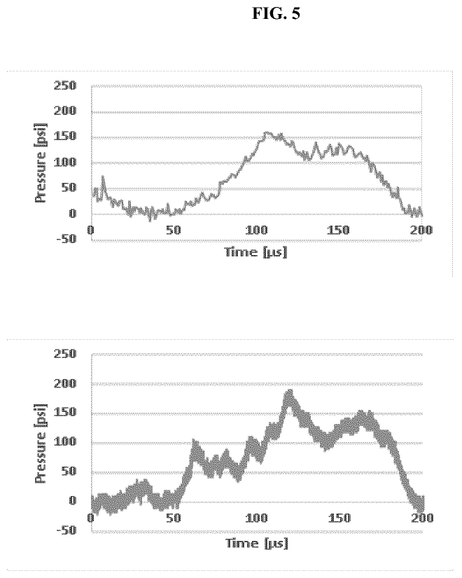

FIG. 5 shows measurement of the blast capsule inside the Hopkinson Bar setup. For this measurement the capsule and the reference sensor's peak height is within 10% of the reference the peak width is within 11% of the reference and the rise time is within 1% of the reference.

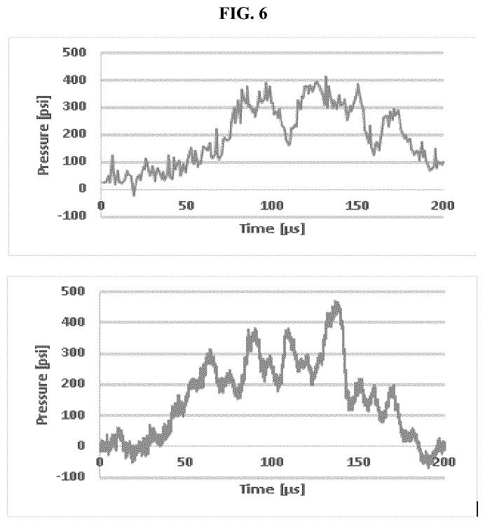

FIG. 6 shows Measurement of the blast capsule inside the Hopkinson Bar setup. For this measurement the capsule and the reference sensor's peak height is within 11% of the reference the peak width is within 7% of the reference and the rise time is within 5% of the reference.

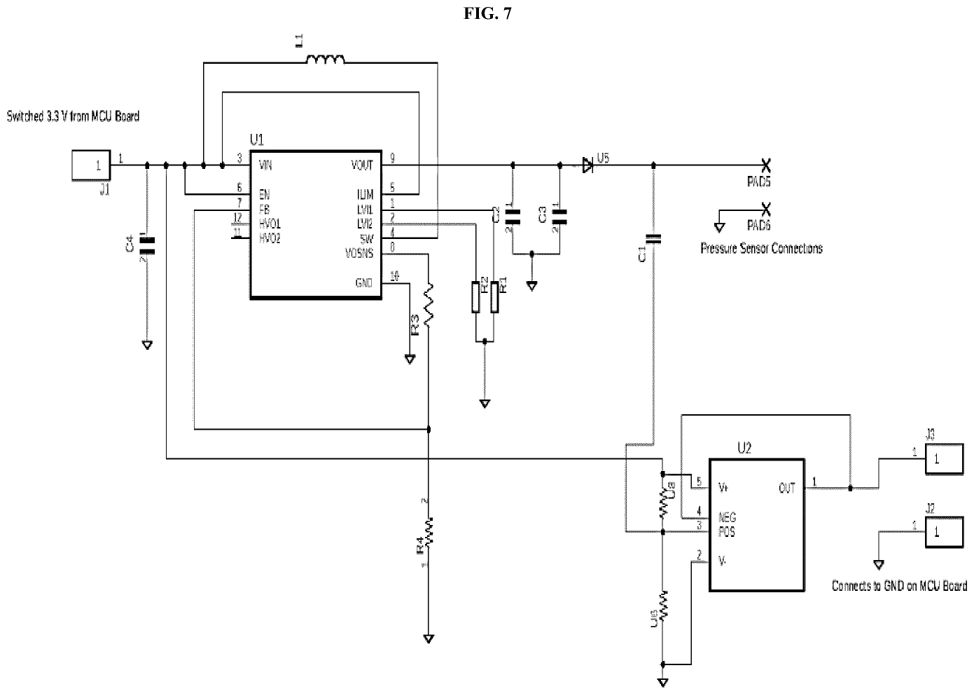

FIG. 7 shows the electric circuit design and required parts for the pressure interface board (pressure sensing module) of the prototype blast capsule.

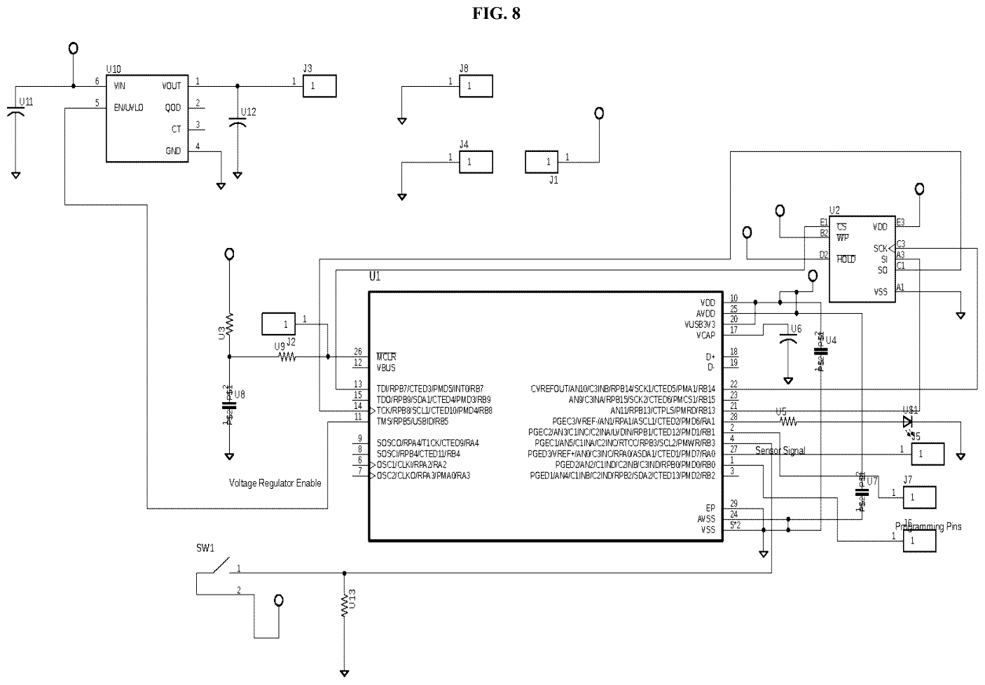

FIG. 8 shows the electric circuit design and required parts for the microcontroller board (electronics module) of the prototype blast capsule.

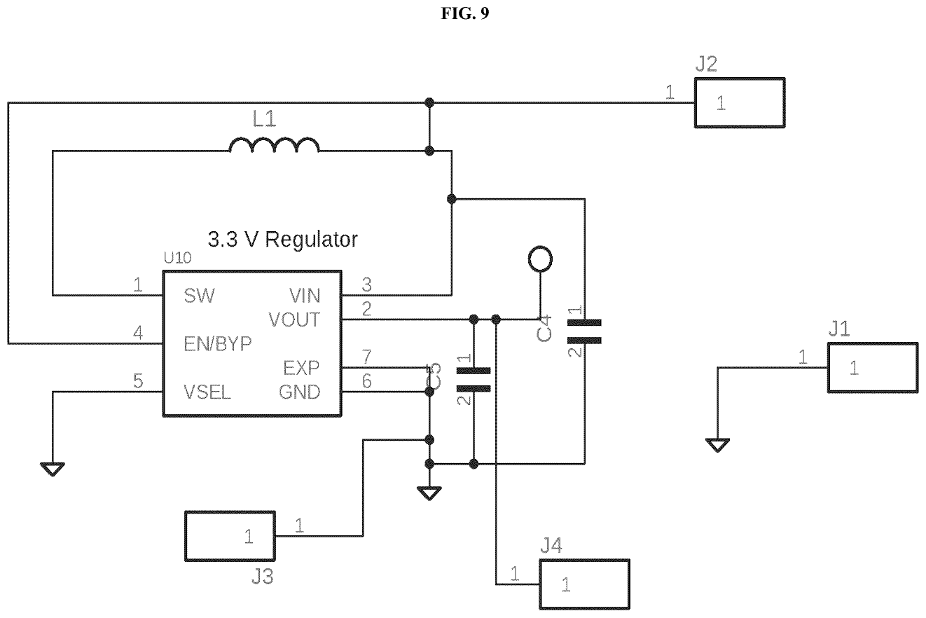

FIG. 9 shows the electric circuit design and required parts for the power supply board (power supply) of the prototype blast capsule.

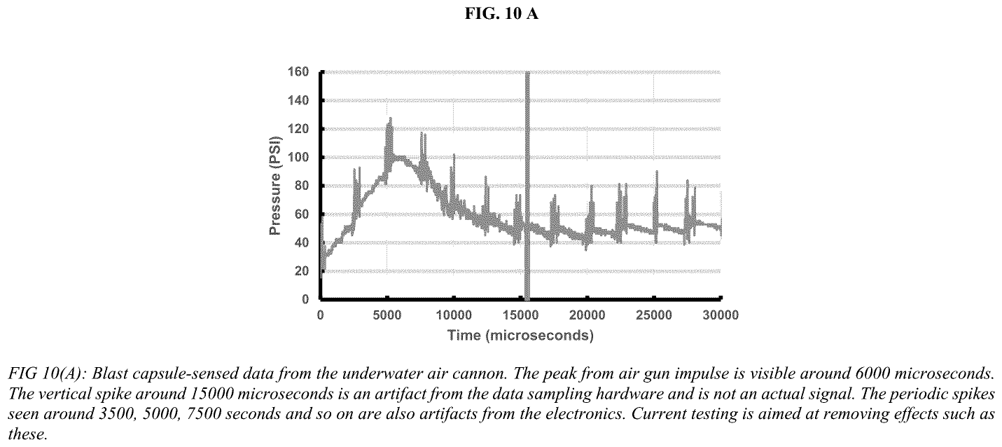

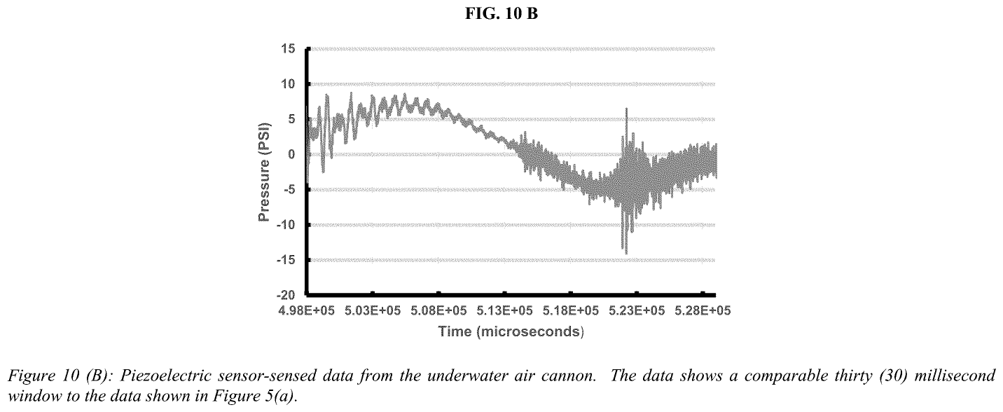

FIG. 10 shows measurement of the blast capsule response to an underwater blast while surrounded with gelatin.

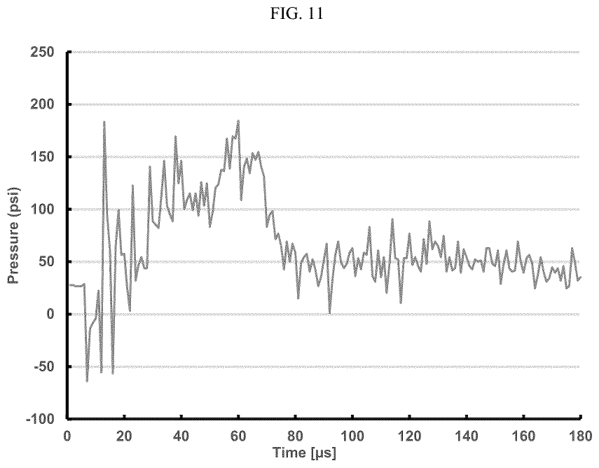

FIG. 11 shows pins connection between pressure interface board, microcontroller board and power supply board when the blast capsuled is assembled. The codes for the parts corresponds to the code listed in Tables 2-4 that are connected when the capsule is assembled are shaded the same.

### DETAILED DESCRIPTION OF THE INVENTION

In the following detailed description, reference is made to the accompanying drawings, which form a part of the specification. It is to be understood that other embodiments may be utilized and structural or logical changes may be made without departing from the scope from the present invention. The following detailed descriptions, therefore, is not to be taken in a limiting sense. In other instances, well-known methods, procedures, and components have not been described in detail so as not to obscure the present invention.

The present invention provides a device and method for monitoring a subject's internal pressure changes as result of a blast. A blast (or blast overpressure) is the pressure caused by a shock wave over and above normal atmospheric pressure as result of an explosion. The initial application is for measuring internal pressure changes from a blast caused by underwater explosion. However, the inventive device may also be used for measuring internal pressure changes due to air blast exposure. The inventive device is designed to be embedded inside a subject, either as an ingestible device or as an implantable device.

FIG. 1A is schematic drawing of the inventive device for blast measurement, which is hereafter referred to as a “blast capsule”. The blast capsule comprises a housing, which contains a 1) pressure sensor capable of measure high frequency pressure wave generated by a blast; 2) a portable power source, such as a battery, which is operatively connected to the pressure sensor and to 3) an electronic module, which comprises of one or more circuit boards, which controls the operation of the blast capsule. As shown in FIG. 2 a housing 11 forming a capsule, which contains within 1) a pressure sensor 12; 2) a power supply 14; and an electronics module 15. The power supply is operatively connected to and powers the pressure sensor 12 and the electronics module 15. The electronic module 15 is operatively connected to and in communication with the power supply 14, and the pressure sensor 12, whereas the electronics module 15 controls the operation of the blast capsule. The electronics module 15 may further include a wireless communication unit. There are several examples of capsule systems with a wireless data transfer capability, two of which are described in G. Traverso et al., “Physiologic Status Monitoring via the Gastrointestinal Tract,” PLOS One, vol. 10, no. 11, p. e0141666, November 2015, doi: 10.1371/journal.pone.0141666. and M. Beccani, E. Susilo, C. Di Natali, and P. Valdastri, “SMAC—A Modular Open Source Architecture for Medical Capsule Robots,” Int. J. Adv. Robot. Syst., vol. 11, no. 11, p. 188, November 2014, doi: 10.5772/59505.

In response to sensed conditions within a digestive tract, the blast capsule 10 is readily ingestible by the subject and can naturally travel within a digestive tract of a human body. The Blast Capsule 10, preferably sized and shaped for the digestive tract, and preferably has a generally rounded, or oblong shape, as shown in FIG. 1A. However, blast capsule 10 can take other sizes and shapes, provided that the shapes and sizes allow the blast capsule to readily passable through digestive tract 34, and is adequately house all the components (11, 12, 14, 15).

FIG. 1B shows the blast capsule 10 in association with human body 30 including digestive tract 32 having mouth 34, esophagus 36, stomach 38, small intestine 40, large intestine 42, and rectum 44. Once ingested within mouth 34, blast capsule 10 may travel the full path of digestive tract 32 until the blast capsule 10 is captured upon exiting at rectum 44. The blast capsule 10 can also be implanted within any of these locations, or subcutaneously. Each of the named locations within the human body represents examples of locations at which the blast capsule 10 sense, and record data regarding internal pressure changes due to a blast. The size of these organs can easily accommodate the inventive blast capsule. The esophagus, which connects the throat to the stomach, is a hollow, muscular tube that for an average adult is approximately 25 cm long, and 1.5-2 cm in diameter. In its deflated state, the stomach contains many longitudinal folds, and at the pylorus, the narrowest part of the stomach, it is about 2 cm in diameter. The small intestine is an elastic lumen that is 3 to 4 cm in diameter, and is the longest portion of the GI tract (approximately 670-760 cm in length). Large intestine or colon measures about 150 cm in length and 6 cm in diameter.

Of course, operation of blast capsule 10 is not limited to the named locations as the sensing, and recording functions of the blast capsule 10 can be performed anywhere within human body 30. For example, those skilled in the art will appreciate that it can be apply to other body lumen or cavities. The term “lumen” as used herein refers to the space within a tubular wall (e.g. a vessel) or the cavity within a hollow organ, such as bladder, uterus, lung, vagina, biliary duct (including bile duct), or blood vessels. The blast capsule may be surgically implanted and affixed within these lumens.

The housing 11 of the blast capsule 10 is preferred to be made of or coated with an inert (i.e. non-digestible) material, so that the blast capsule 10 passes through digestive tract without being consumed, which may include Teflon, glass, ceramic and other materials known to those skilled in the art. Other biocompatible materials may be also used for the housing of the blast capsule if it is to be implanted within a subject. Example of these biocompatible materials include but not limited to Alumina, Bioglass, Cobalt-chromium alloy, Hydroxyapatite, Medical-grade silicone (short-term implantable and long-term implantable), Polyvinylchloride (PVC), Polyethylene (PE), Polypropylene (PP), Polytetrafluoroethylene (PTFE), Polymethylmethacrylate (PMMA), Stainless steel, Trimethylcarbonate (polymer), TMC NAD-lactide, Titanium and titanium alloys, Zirconia.

The housing 11 is waterproof to protect the components from fresh or seawater (when used as a wearable blast sensor) or bodily fluids (when used as an ingestible or implantable blast sensor). The materials used for encapsulation must possess favorable mechanical properties so that the pressure waves can travel through it to reach the pressure sensor.

The electronics module 15 receives and records the blast related pressure data within the subject and regulates the functions of the pressure sensor 12. The electronics module 15 can transmit the recorded information to a computer or an electronic device via wired or wireless connection. In a preferred embodiment, as illustrated in FIG. 2, the electronic module may comprise a microcontroller (MCU), which controls the functions of the power supply and the pressure sensor; and a state machine that is used to operate the MCU. It may further comprise supporting electronics to allow the smooth operations of these components. The electronic module 15 is operatively connected to the pressure sensor 12 and the powers supply 14.

The algorithm that runs the blast capsule is described in the state diagram shown in FIG. 3. There are five main states, each with a specific function. The transitions between the five states are controlled either by specific inputs or, in some cases, automatically. In the prototype blast capsule, the state transition is coded with the help of the MPLAB X development environment for PIC32 MCUs. When the embedded system first turns on, it enters the Data Transfer State where the MCU reads the data back from the ferroelectric random access memory (FRAM) using the serial peripheral interface (SPI) and then sends the data to a computer using the universal asynchronous receiver transmitter (UART). A computer is able to read the data by using a UART to universal serial bus (USB) bridge.

At the completion of the data transfer, the MCU enters the Initial Sleep mode, which is a low power state. The target current consumption for this state is less than 200 μA. The purpose of this state is that is conserves power and it is meant to be the state the devices stays in between the time that the electronics are encased in the waterproof polymer and when it is turned on to start data collection. FIG. 2 shows that a magnetic reed switch that may be used to trigger a comparator interrupt in the MCU waking the device from this Initial Sleep State. The reed switch can be actuated by waving an external magnet next to the capsule. In alternative design, other suitable activators can also be used in place of a reed switch. It is preferred to have an activator, because it allows packaging long before use and give some control over the wake time. There is an alternate version of the blast capsule that was created where the wake from sleep is timed and must be set before the system is packaged, though this version is less desirable.

After the device is awoken using an activator such as reed switch/comparator interrupt it enters another Short Sleep State. Again, the target current consumption for this state is less than 200 μA. The purpose of the Short Sleep State is to wake up the system from the Initial Sleep State, and then wake again at a shorter predictable interval. For example, if one packaged the blast capsule one to two days before testing. The blast capsule would enter the Initial Sleep State for those one to two days, be awoken by the activator at the data collection site on the morning of the test and then go to Short Sleep again for 150 minutes (or other pre-specified amount of time). The MCU wakes up the blast capsule from the Short Sleep State using a timer interrupt and enters the Data Recording State. In the Data Recording State, the MCU is sampling data from the pressure sensor at 1-2 megasample per second (1-2 MSPS). If a peak is detected, the MCU will write the data to the FRAM using the SPI and enter the Deep Sleep State. If no peak is detected, the MCU will continue sampling data until it runs out of battery, which is depicted as Deep Sleep in the Sate Diagram.

Once the task of measurement/monitoring is completed, the data can be recovered by demolding the MCU from the housing in which it is encased and then powering the MOU from a power supply. Once powered, the MCU will enter the Data Transfer State and begin to transfer the data from the FRAM to a PC or other electronic device over the UART interface (with the MCU passing the data). Other suitable wired or wireless data transmission interface can also be used for data transfer.

Pressure Sensor 12 of the blast capsule 10 senses internal pressure and pressure changes due to a blast. As used herein, a “blast” refers to an explosion or a high pressure wave often accompanied by high temperature, such as during an explosion of an improvised explosive device (IED). It is connected to a power supply (i.e., a battery) and the electronic module 15. To perform its designed function, the pressure sensor 12 may be adapted to gather blast data and to provide the blast data the electronics module 15 to be recorded. The suitable pressure sensor 12 must be able to detect high pressure events, such as pressure waves accompanying an explosion or blast, which are characterized by a rapidly moving pressure/shock wave (often exceeding ˜330 meters per second in air) with a blast overpressure (i.e. magnitude of the moving pressure wave measured as a pressure difference over and above a normal atmospheric/ambient pressure) which can be several atmospheres greater than ambient pressure. In an embodiment, the pressure sensor 12 must be able to sample signals at frequency as high as 1 megasample per second (MSPS). The pressure sensor 12 are selected based on following design goals: (1) low power operation including operation from a voltage that can be generated from battery, (2) a compact form factor appropriate for a swallowable application, (3) the ability to withstand molding during capsule assembly, and (4) the ability to withstand and measure blast energy. In prototype device, as shown in FIG. 2, a Sensor Interface Board is used with the pressure sensor 12. The sensor interface board contains an 18 V boost regulator, which takes the 3.3 V system voltage, and boosts it to 18 V that is needed to operate the pressure sensor 12. The sensor Interface Board also contains a diode and capacitor circuit needed to feed the 18 V boost regulator voltage into the pressure sensor. A piezoelectric pressure sensor is attached directly to the Sensor Interface Board using PC pins. An op-amp buffer on the Sensor Interface Board buffers the sensor signal as it goes back to the microcontroller. The sensor signal goes through the op-amp buffer into one of the MCU's analog to digital converter (ADC) input pins where the analog output signal from the sensor is digitized. When the system is in any state other than the Data Recording state, the switch on the MCU board is turned off so that none of the components on the Sensor Interface Board receives any power, thus reducing the current draw on the battery. When the system enters the Data Recording state, the switch on the MCU board powers the Sensor Interface Board, allowing it to operate.

In an embodiment, a peak detection algorithm is used for the blast capsule and works as follows. The data from the ADC is stored in two different buffers, which are constantly flip-flopping. The buffers are stored in the MCU's random access memory. While one buffer is being filled with data, the data in the second buffer are being analyzed. A loop is used to go through the buffer and see if any of the data exceed a pre-set threshold. If the threshold is exceeded, an IF statement is used to set a flag. Once the buffer that is not being screened is done filling, the flag causes the data collection to cease and the data are written to FRAM as described above. Persons with ordinary skills in the art would appreciate other alternative algorithms, which can also be applied for blast detection, such as algorithms that are immune to electronic or environmental noise.

The application also describes a method for continuously monitoring internal pressure changes experienced by a subject during a blast. First, a blast capsule as previously described is provided. Before the subject is exposed to a blast, the subject is asked to swallow the blast capsule. The blast capsule is activated. During the blast, internal pressure data related to the blast is automatically measured and recorded by the blast capsule. After the occurrence of the blast, the blast capsule is allowed to naturally pass through the digestive tract and collected. Internal pressure data associated with the blast is then downloaded for analysis via the blast capsule's wired or wireless connection portal. Alternatively, the blast capsule can be surgically implanted and affixed inside any suitable body cavities within the subject, including but not limited to mouth, esophagus, stomach, small intestine, large intestine, and rectum. The blast capsule is activated before the expected exposure to a blast, which will automatically measure and record internal pressure changes due to the occurrence of a blast. After the occurrence of a blast, the blast capsule can be surgically removed from the subject. Internal pressure data associated with the blast is then downloaded for analysis via the blast capsule's wired or wireless connection portal.

### EXAMPLE 1: PROTOTYPE BLAST SENSOR

### 1. Circuit Design and Hardware

An overview of the blast capsule is shown in FIG. 2 and major components are shown in Table 1.

### A. Microcontroller and State Machine

The blast capsule is operated by a state machine that runs on a 32-bit PIC microcontroller (MCU). The PIC32MX274F256B microcontroller (Microchip, Chandler, AZ USA) was chosen for several reasons including: (1) a large amount of onboard data memory, (2) the ability to sample the ADC at or above 2 MSPS, (3) it can be powered from as low as 2.5 V, (4) it has an acceptable current consumption when sampling at 1 MSPS (20 mA) as well as robust low power modes that can be used in a sleep state, and (5) it is available in a package with an acceptable form factor (6 mm by 6 mm QFN package). The combination of these features made this MCU well suited for application in the blast capsule.

The state machine contains five main states, each with a specific function. The state transitions either are handled by specific inputs or, in some cases, occur automatically. When the embedded system first turns on, it enters the Data Transfer state (1) where the MCU reads the data back from the ferroelectric random access memory (FRAM) using the serial peripheral interface (SPI). The MCU then sends the data to a computer using the universal asynchronous receiver transmitter (UART). A computer can read the data by using a UART-to-universal serial bus (USB) bridge. (This data transfer is actually used at the end of the measurement sequence when the capsule is demolded, and the data are read back to a computer.) (2) At the completion of the data transfer, the MCU enters the Initial Sleep mode, which is a low power state. The purpose of this state is to conserve power between the time that the electronics are encased in the biocompatible polymer and when the capsule is turned on to start data collection (the target current consumption for this state is less than 200 μA.). A magnetic reed switch is used to trigger a comparator interrupt in the MCU waking the device from this Initial Sleep state (the switch is tied to 3.3 V and pulls the pin to which it is connected high when triggered by an external magnet). This is the preferred method because it allows packaging long before use and some control over the wake time. (3) After the device is awakened using the reed switch/comparator interrupt, it enters another Short Sleep state. This is another low power state with a target current consumption of less than 200 μA. The purpose of this state is to allow the system to wake up from the initial sleep, and then wake again after a shorter, predictable interval. For example, if the capsule is packaged one to two days before testing it could enter the Initial Sleep state for those one to two days, be awoken by the comparator interrupt at the data collection site on the morning of the test and then go to sleep again for 150 minutes. (or another pre-specified amount of time) before actual use in an experiment. (4) When the MCU wakes up from the Short Sleep (using a timer interrupt), it enters the Data Recording state. In the Data Recording state, the MCU is sampling data from the sensor at 1 MSPS. If a pressure peak is detected (please see below for an explanation of the peak detection algorithm), the MCU will write the data to the FRAM using the SPI and enter a (5) deep sleep state from which it will not awake. If no peak is detected, the MCU will continue sampling data until it runs out of battery. Once the experiment (or measurement/monitoring) is completed, the data can be recovered by demolding the system from the polymer in which it is encased and then powering the system from a power supply. Once powered, the system will enter the Data Transfer state during which it transfers the data from the FRAM to a PC over the UART interface using a UART to USB bridge (with the MCU passing the data).

### B. Pressure Sensor and Supporting Electronics

The overview of the blast capsule hardware are shown FIG. 2 and table 1.

TABLE 1
Description of the major Components.

| Component Name | Manufacturer | Part Number | Description |
|---|---|---|---|
| Microcontroller | Microchip | PIC32MX274F256B | 32-bit low power |
| | | | microcontroller |
| FRAM | Fujitsu | MB85RS1MTPW-G-APEWE1 | Non-volatile |
| | | | memory |
| Reed Switch | Standex-Meder | MK24-A-3 | NO Magnetic Reed |
| | | | Switch |
| 3.3 V Regulator | Texas | TPS61291DRVR | 3.3 V Boost |
| | Instruments | | Regulator |
| 18 V Regulator | Texas | TPS61096ADSSR | 18 V Boost |
| | Instruments | | Regulator |
| Power Switch | Texas | TPS22810DRVT | Power Switch |
| | Instruments | | |
| Op-Amp | Texas | LM8261M5 | Op-Amp Buffer |
| | Instruments | | |
| Battery | Energizer | 2L76 | Lithium Ion Battery |
| Pressure Sensor | PCB | 138M190 | Miniature Blast |
| | Piezotronics | | Pressure Sensor |

The necessary components are divided among three four-layer printed circuit boards, including the power board, data board, and sensor interface board. The boards connected together using PC pins and PC pin receptacles, and circuit design and hardware required for each board are shown in more details in FIGS. 7, 8, 9 and Tables 2, 3, 4.

TABLE 2
Part List for Sensor Interface board

| Code | Manufacturer | Part # | Part Name |
|---|---|---|---|
| U2 | Texas | LM8261M5 | Op-Amp |
| | Instruments | | |
| U5 | Microsemi | CMJ2000TRSOD-123 | Current Limiting Diode |
| J1, J2, J3 | Mill Max | 3016-0-15-15-21-27-10-0 | PC Pin Receptacle |
| C1 | Samsung | CL21A226MAQNNNE | 22 uF Capacitor |
| U8 | Yageo | RC0201FR-07124KL | 124K Resistor |
| U6 | Yageo | RC0201FR-0711KL | 11K Resistor |
| C4 | Murata | GRM035R60J475ME15D | 4.7 uF Capacitor |
| U1 | Texas | TPS61096ADSSR | Boost Switching |
| | Instruments | | Regulator |
| R4 | Yageo | MCS04020C1002FE000 | 10K Resistor |
| R1, R2 | Yageo | RC0603FR-071ML | 1M Resistor |
| C2, C3 | Murata | ZRB18AR61E106ME01L | 10 uF Capacitor |
| R3 | Panasonic | CRCW0603169KFKEA | 169K Resistor |
| L1 | Wurth | 74479299222 | 2.2 uH Inductor |
| (Unlisted) | PCB | 138M190 | Pressure Sensor |
| | Piezotronics | | |

TABLE 3
Part List for Microcontroller (Data) Board

| Code | Manufacturer | Part # | Part Name |
|---|---|---|---|
| J1-8 | Mill Max | 3016-0-15-15-21-27-10-0 | PC Pin Receptacle |
| U1 | Microchip | PIC32MX274F256BT-I/MM | PIC32 MCU 28-VQFN |
| U2 | Fujitsu | MB85RS1MTPW-G-APEWE1 | SPI FRAM |
| U10 | Texas | TPS22810DRVT | Power switch |
| | Instruments | | |
| U5 | Vishay Dale | CRCW02013K00FKED | 3K Resistor |
| U6 | AVX | TLCK106M006QTA | 10 uF Capacitor |
| U11-12 | Samsung | CL03A105MP3NSNC | l uF Capacitor |
| U4, U7-8 | Yageo | CC0201KRX5R7BB104 | . 1 uF Capacitor |
| U$1 | Kingbright | APG0603SEC-TT | Orange LED |
| U3 | Yageo | RC0201JR-079K1L | 9K Resistor |
| SW1 | Standex-Meder | MK24-A-3 | SPST-NO Reed Switch |

TABLE 4
Part List for Power Board

| Code | Manufacturer | Part # | Part Name |
|---|---|---|---|
| J1-4 | Mill Max | 3016-0-15-15-21-27-10-0 | PC Pin Receptacle |
| C4, C5 | Murata | GRM21BR60J107ME15L | 100 uF Capacitor |
| L1 | Taiyo Yuden | MDMK2020T3R3MMV | 3.3 uH Inductor |
| U10 | Texas | TPS61291DRVR | 3.3 V Boost Regulator |
| | Instruments | | |

As mentioned, there are a few variations of the design. This discussion will focus on the design that uses a reed switch to wake up the system, as it is the most versatile version. However, persons with ordinary skills in the art would appreciate another alternative designs that can also be applied for blast detection. The Power Board (FIG. 9) for the blast capsule contains a 3.3 V regulator that steps the battery voltage up from the nominal 3V value from the 2L76battery to 3.3 V. The boost regulator on this board also holds the system voltage at 3.3 V even as the battery voltage begins to drop as the battery discharges. The MCU Board, or Data Board (FIG. 8), contains the microcontroller, the FRAM, the reed switch and a power switch. The FRAM and reed switch have been discussed above. The power switch is used to control the flow of power to the Sensor Interface Board (FIG. 7). A light emitting diode (LED) on the MCU Board lights up whenever the system is active. The Power Board for the blast capsule contains a 3.3 V boost regulator to step up the nominal 3V 2L76 (Energizer, St. Louis, MO USA) battery voltage. The Sensor Interface Board contains an 18 V boost regulator that steps the 3.3 V system voltage up to 18 V, which is the voltage needed to operate the pressure sensor. The Sensor

Interface Board also contains a diode and capacitor circuit needed to feed the 18 V boost regulator voltage into the pressure sensor (per the manufacturers application note). The piezoelectric pressure sensor is attached directly to the Sensor Interface Board using PC pins. The pressure sensor has a form factor of 4 mm by 4 mm and pressure range of 200 psi, a resolution of 1 psi, and a sensitivity of 2.5 mV per PSI. The sensor signal goes through an op-amp buffer on the Sensor Interface Board into one of the MCU's analog to digital converter (ADC) input pins where the analog output signal from the sensor is digitized. The op-amp buffer is setup so that it also provides a small DC offset for the sensor signal. This allows the system to measure small negative pressure swings (the ADC on MCU is single ended).

### C. Peak Detection Algorithm

The peak detection algorithm for the blast capsule works as follows. The sensor data read by the ADC are stored in two different buffers on the MCU's random access memory (RAM), which are constantly flip-flopping. While one buffer is being filled with data, the data in the second buffer are being analyzed. A loop with an IF statement is used to compare each guffer value to a set threshold (i.e. IF (buffer_value>threshold). If the data exceed the pre-set threshold, a flag is set. To provide some immunity to environment noise, the IF statement condition can be adjusted so that multiple (i.e., 5-10) values that exceed a given threshold need to be detected. If a peak has been detected, once the buffer that is not being screened is done filling, the flag set by the IF statement causes the data collection to cease and the data are written to FRAM using the SPI interface. More complex algorithms are also possible which re more immune to electronic or environmental noise.

The goal of this initial design iteration was to capture between 10-30 milliseconds of blast data for analysis. A single blast peak has a width of 5-10 milliseconds, but there are additional features that can be useful both before and after the peak. The PIC32 MCU that was selected for this work was chosen, in part, due to its large onboard RAM (64 kilobytes (kB)). The ADC on the PIC32 uses 10-bits, thus each data word is 2-bytes. This gives a maximum data sample size of 32 kB. Some RAM is allocated for other functions and the current design was able to capture approximately, 30,000 data words (approximately 30 milliseconds worth of data with a sampling rate of 1 MSPS). The FRAM holds 128 kB worth of data. For some of the experiments described below the algorithm was altered so that each time the blast capsule was molded the pill

### 2. System Packaging

The prototype Blast Capsule is molded into an approximately 12.5 mm diameter (at the widest point) by 31 mm long form factor using 3D printed molds. Polydimethylsiloxane (PDMS) was chosen as the material of choice to mold the capsule because of its ease of curing at 60° C., which is a processing temperature that is also compatible with the electronic components and the battery. PDMS is also biocompatible and readily removable, which allows access to the boards for data retrieval or reconfiguring of components. The size of the capsule is suitable for swallowing or implanting under the skin.

The molding process begins with the assembly of the blast capsule circuit boards and sensor. The boards are stacked and connected by securing PC pins into the appropriate board mounted receptacles. Between each board electrician tape is used as an electrical insulator, preventing the device from shorting. Once the boards are assembled the sensor is connected to the sensor board. Because the sensor leads are sensitive and easily damaged, the sensor is set and cured in a small block of PDMS, prior to being added to the blast capsule assembly. Next, the assembled device is connected to a 2L76 battery. This battery provides 3.0 v (which is stepped up to 3.3 V by the boost regulator on the power board, and is an ideal shape and size for packaging. The 2L76 battery seats in the mold beneath the assembled boards and powers on the blast capsule, this change of state is indicated by an orange LED switching on. After approximately 1 minute the blast capsule enters its low power state (sleep state). When entering a low power state the LED shuts off. Once asleep the blast capsule is loaded into the bottom half of a 2-part mold 3D printed from acrylonitrile butadiene styrene (ABS plastic) material. An O-ring is fitted into a slight groove that surrounds the blast capsule then the top half of the mold is set and secured. The bottom half of the mold is outfitted with 10 thermoplastic inserts that allow 10 screws to join the top and bottom halves of the mold. The mold is fastened and PDMS is injected into the mold via luer lock tipped syringe through an opening in the top half of the mold. The mold is filled then placed in a vacuum desiccator, where air is evacuated from the PDMS. This degassing process takes, on average, 30 min. At the conclusion of its degassing the mold and blast capsule are placed in an oven set to 60° C. PDMS cures in approximately 1 hr at 60° C. Once fully cured the blast capsule is extracted from its mold and any excess PDMS is carefully trimmed with a razor blade.

### EXAMPLE 2: TESTING OF PROTOTYPE BLAST CAPSULE

Multiple tests of varying design were used to validate and calibrate the blast capsule. FIG. 4 illustrates the testing set up. In each test, the blast capsule was accompanied by a reference sensor, and exposed to a pressure generating device. Pressure readings captured by the blast capsule in each test were compared against the reference sensor. Data features such as pressure peaks, peak rise times, and peak width were compared in order to validate measurements captured by the blast capsule.

The Hopkinson bar experiments involved three major components contributing to the testing setup; a cylindrical aluminum canister filled with water, a Hopkinson bar, and a gas fired projectile. The aluminum canister was constructed of a tube and two sealing caps, a top and bottom cap. The tube possessed a length of 24 in an inner diameter of 3.75 in and an outer diameter of 4 in. The canister's top cap had a thickness of 1.25 in and the bottom cap had a thickness of 1.875 in. An O-ring allowed each cap to effectively seal when fitted to the tube. In preparation of each experiment the canister top cap, or lid, was removed so that the reference sensor and blast capsule mount could be installed. The reference sensor possessed a ⅛″ NPT male thread and mated to the canister lid's ⅛″ NPT female threaded port. The blast capsule mount also possessed a ⅛″ NPT male thread and mated to a ⅛″ NPT female port. Before sealing the canister water was refilled so that little to no air existed within the canister during testing. Once the canister was sealed it was secured horizontally to a rail that positioned the Hopkinson bar to be concentric and flush with the canister's bottom cap. The canister was secured in such a way that it would not move upon receiving an impact. Throughout the Hopkinson bar experiments the blast capsule was stationed within the aluminum canister and secured to a small rod fixed at the end of the canister opposite of the Hopkinson bar. The blast capsule was positioned to be level with the reference sensor and facing the pressure wave that would be generated by the gas fired projectile impacting the Hopkinson bar. The gas fired projectile was housed within a steel tube positioned to be concentric with the Hopkinson bar. This steel tube served as a track for the projectile. The projectile was fired by compressed air at a pressure ranging from 75 psi to 100 psi. Upon the projectile's impact a pressure wave would propagate through the Hopkinson bar then travel through the water filled canister eventually reaching the blast capsule and reference sensor. Data related to the pressure wave are captured by both devices and compared against one another.

The explosive exposure experiments, subjected the blast capsule to a pressure wave generated by a small, electrically excited, explosive device. The explosive device as well as the blast capsule and reference sensors were submerged in a cubic tank filled with approximately 307 gallons of water. The blast capsules were stationed within mesh bags that were suspended by common fishing line, to be level with the explosive. The mesh bags were weighted by washers and positioned to be 8 in radially away from the explosive device. Throughout these experiments blast capsules were tested two at a time and accompanied by a reference sensor. Once all necessary devices were positioned the blast capsules were triggered by magnet and a stopwatch timer was simultaneously started. The blast capsules were programmed to wait approximately 30 mins after being triggered to wake up. This delay accounts for the time it takes to fill the testing tank with water. Typically the blast capsules fully activated approximately 10mins after the testing tank reached its target volume. Viewing ports installed in the tank allowed for potential visual confirmation of blast capsule activation, however the mesh bags obscured the devices to a degree. Once the blast capsules activated approximately 2.5 mins passed before firing the explosive. At the conclusion of this experiment the tank's water was drained and the blast capsules were collected from their mesh bags. Data was extracted from the blast capsule shortly after being removed from the testing setup.

The initial underwater testing was completed in two phases:

The first phase was intended to ensure that the PDMS mold created a water-tight seal by molding the complete circuit and placing it in water for an eighteen (18) hour overnight test. Following the full sleep duration, the capsule successfully woke and recorded new drop test data, similar to the data above, which verified the integrity of the molded capsule. This test was repeated several times in order to verify repeatability and molding consistency. The results of the test further proved the repeatability of the drop test method, resulting in consistent data capture.

The second phase of testing was performed using the Hopkinson bar setup described above and shown in FIG. 4. These tests were performed to look at the repeatability and accuracy of the blast capsule captured data as comparted to a known reference sensor. In the Hopkinson bar setup it's easier to create a propagating pressure waveform without motion artifacts, which is a major issue with the air cannon setup. The data captured from the Hopkinson is shown in FIG. 5 and FIG. 6. The data looks comparable to the reference sensor with the small differences explained by differences in the way the sensors are packaged (the reference sensor is not embedded in polymer for example) as well as small differences in orientation. In comparing metrics for the blast pill data it was as close as 10-11% to the references sensor in terms of peak pressure, within 1-5% of the rise time measured with the reference sensor and within 7-11% of the measured peak width. These data show that the blast capsule is able to capture underwater pressure wave data (from a simulated pressure pulse or blast) that is comparable to that of a commercial underwater blast sensor.

The third phase tested the circuit when exposed to an underwater explosion with a peak pressure of approximately 190 pounds per square inch (PSI). The molded capsule was tied with fishing line from a bar, weighed down for security by a suspended 20 gram weight, and placed one (1) meter away from the blast source. The data recorded by the blast capsule were compared against data from two blast sensors that were suspended in a similar way. The results demonstrated that the capsule could successfully complete an overnight long-sleep, wake-up with the comparator actuation, a short-sleep for another two (2) hours, and then record a live underwater blast.

Two additional underwater blast experiments were carried out in an open air rectangular tank measuring 4 feet on a side filled with approximately 307 gallons of water. The explosive exposure experiments, subjected the blast capsule to a pressure wave generated by a small, electrically excited, explosive device. The blast capsules were stationed within mesh bags that were suspended by common fishing line, to be level with the explosive. The mesh bags were weighted by washers and positioned to be 8 in radially away from the explosive device. In the first experiment, the blast capsule was left open in the tank. The peak pressure that was measured by the blast pill was within 9% of the data measured by a reference sensor. Due to the proximity of the blast capsule to the blast there is a second peak seen in the blast capsule data due to a reflection off the back plate of the sensor, which was not seen in the reference sensor data.

After the Hopkinson bar experiments were completed to show that the device could adequately capture blast data underwater, the capsule was exposed to underwater blasts as described above. The capsule was exposed both directly and while inserted into a bag of gelatin. The direct measurements showed that the capsule could function in the underwater environment and the recorded peak pressure was comparable to a reference sensor. This direct measurement, however, showed a reflection of the blast wave from the substrate where the transducer was mounted, which made the data more difficult to interpret compared to the reference sensor. In an additional test, the blast capsule was inserted into a bag of gelatin and then exposed to an underwater blast, which likely is a more realistic reflection of the capsule if it were ingested prior to exposure. The data from this measurement are shown below in FIG. 10. For this measurement, a reference sensor was not included, but the previous calibration using the Hopkinson bar setup was used to benchmark the data.

### Future Studies

This initial development effort has demonstrated a functional ingestible device that measures internal pressures from high-energy acoustic energy and blast. Further development of this ingestible system for animal experiments and eventual use in humans will fill the knowledge gap regarding how blast affects internal organs. Furthermore, this ingestible device could be used in the field in high-risk individuals to provide lifesaving diagnostic information to first responders. The capsule or a similar design could also find potential application of a small form factor embedded external blast monitor.

Although we chose to conduct our initial testing in the liquid environment, the blast capsule could also be used for in-air applications. The capsule showed good agreement with a reference sensor in the test setup.

This prototype blast capsule was assembled by hand in the lab. The assembly reliability could be improved readily with industry-standard equipment and manufacturing practices. Additionally, lower voltage pressure sensors are available that could greatly improve the battery performance of the system. Reducing the current draw of the 18 V regulator could be accomplished using lower voltage sensors. In addition, a low current draw could allow other battery technologies with a higher amp hour capability (but lower maximum current capabilities), such as lithium thionyl chloride batteries. The 2L76 battery can source up to 170 mA of current, which is one of the reasons that it was chosen.

Furthermore, the form factor (particularly the diameter) could be reduced by molding the pill using epoxy. Biocompatible epoxies are readily available for endoscopy and surgical equipment. Epoxy is more rigid and less likely to de-bond from the underlying structure, and a thinner layer of encapsulant could be used to reduce the diameter of the capsule. Future work should explore a more robust peak detection algorithm that could make the system more immune to environmental noise and false triggering. One solution is to incorporate additional electronic filtering in front of the ADC to block signals with different frequency band than the blast signals.

This prototype shows that the outlined design specifications are achievable in an ingestible format. Moving this system beyond the initial prototype stage could open a variety of applications in the development of standoff guidance standards as well as potentially combat casualty care. The eventual goal is to apply the blast capsule to human or combat casualty care applications. In particular, in the area of injury prediction on the battlefield. For example, the blast capsule device could be used to screen for high energy blast exposure, and then use that data to risk stratify individuals based on injury probability (based on the measured blast dose), which could be used to prioritize patients based on need of care.

The ingestible system is superior to wearable or helmet mounted blast sensors. For experimental applications, a fixed blast sensor can be used, but limits the information one can gather because the sensor may not be in the same location as the test subject when the blast occurs. Furthermore, externally mounted either wearable or fixed position blast sensors do not give the same information on internal blast pressures achieved as would be reported by an ingestible system.

What is claims is:

1. A blast capsule for monitoring internal pressure experienced by a subject from a blast, said capsule comprising a housing having
a) a power supply;
b) a pressure sensor contained adapted to measure overpressure caused by a blast; and
c) an electronics module operatively connected to the power supply and the pressure sensor;
whereas said electronics module controls the operation of the blast capsule and records pressure data taken by said pressure senor.

2. The blast capsule of claim 1, wherein said capsule is sized and adapted to be ingested by or implanted inside the subject.

3. The blast capsule of claim 2, wherein said housing is made of an inert material.

4. The blast capsule of claim 2, wherein said housing is waterproof.

5. The blast capsule of claim 1, wherein said pressure sensor is capable of measuring high energy and high frequency pressures associated with a blast.

6. The blast capsule of claim 5, wherein said pressure sensor is measuring pressure at a frequency at least 1 megasample per second (1 MSPS).

7. The blast capsule of claim 1, wherein said electronics module is designed to operate with a low power consumption.

8. The blast capsule of claim 1, wherein said electronics module may further contains an activation switch, which turn on the blast capsule.

9. The blast capsule of claim 1, wherein said electronics module switch blast capsule into a sleep state during non-recording period to conserve power.

10. The blast capsule of claim 1, wherein said electronics module records measurements provided by said pressure sensor and transmits the measurements to a computer via wire or wireless communications.

11. The blast capsule of claim 1, wherein said power supply comprises a battery.

12. A method for continuous monitoring internal pressure changes experienced by a subject during a blast, comprising:
a) providing a blast capsule of claim 1;
b) having the subject ingest or implanting inside a subject a blast capsule of claim 1;
c) activating said blast capsule
d) measuring and recording internal pressures inside the subject during a blast or explosion; and
e) transmitting said measurements to a remote computer or electronic device.

13. The method of claim 12, wherein said electronics module is programed to control the sampling rate of said sensor.

14. The method of claim 12, wherein said electronics module is programmed to control a transmission burst duration and a rate of transmission bursts of said transmitter.

15. The method of claim 12, wherein said electronics module is programmed to activate the blast capsule to record pressure data inside the subject.

16. The method of claim 13, wherein said blast capsule measures and records internal pressures inside the subject at a sampling rate of 1 at least 1 megasample per second (1 MSPS).
